(19) United States
(12) Patent Application Publication
(10) Pub. No.: US 2021/0353967 A1
(43) Pub. Date: Nov. 18, 2021

(54) METHODS AND SYSTEM FOR SELECTIVE AND LONG-TERM NEUROMODULATION USING ULTRASOUND

(71) Applicant: CARNEGIE MELLON UNIVERSITY, Pittsburgh, PA (US)

(72) Inventors: Kai Yu, Pittsburgh, PA (US); Xiaodan Niu, Pittsburgh, PA (US); Bin He, Pittsburgh, PA (US)

(73) Assignee: CARNEGIE MELLON UNIVERSITY, Pittsburgh, PA (US)

(21) Appl. No.: 17/284,244

(22) PCT Filed: Oct. 11, 2019
(86) PCT No.: PCT/US2019/055955

§ 371 Date: Apr. 9, 2021

Related U.S. Application Data
(60) Provisional application No. 62/766,306, filed on Oct. 11, 2018.

(51) Int. Cl. A61N 7/00; A61B 5/291; A61B 5/00; A61B 5/256; A61B 5/369; G16H 20/40

(52) U.S. Cl. CPC A61N 7/00; A61B 5/291; A61B 5/4836; A61B 5/256; A61B 2562/046; G16H 20/40; A61N 2007/0026; A61N 2007/0056; A61B 5/369

(57) ABSTRACT

Specific parameter sets are provided that makes the transcranial focused ultrasound to selectively activate a certain neuronal type at cortical brain and enables the transcranial focused ultrasound to non-invasively induce long-term effects at deep brain. A type of ultrasound collimator with incidence angle control is designed and validated through acoustic field pressure mapping in order to target brain areas at different depths. Multi-elements transducer arrays are also used to achieve transmission of focused ultrasound.

### CROSS-REFERENCE TO RELATED APPLICATIONS

This application claims the benefit under 35 U.S.C. § 119 of Provisional Application Ser. No. 62/766,306, filed Oct. 11, 2018, which is incorporated herein by reference.

### STATEMENT REGARDING FEDERALLY SPONSORED RESEARCH

This invention was made with government support under NIH-7RF1MH114233 awarded by the National Institutes of Health. The government has certain rights in this invention.

### BACKGROUND OF THE INVENTION

The invention relates to methods and a system of using ultrasound for neuromodulation. More specifically, the invention relates to methods and a system of using transcranial focused ultrasound (“tFUS”) to stimulate different neuron types and modulate synaptic connectivity in living subjects and to induce sustained neural effects after the cessation of tFUS.

Neuromodulation is a technique to intervene with the nervous system in an attempt to improve the quality of life of subjects suffering from neurological disorders. For decades, a myriad of brain neuromodulatory approaches, such as deep brain stimulation, transcranial magnetic stimulation, transcranial current stimulation, transcranial focused ultrasound, transcranial static magnetic field stimulation, optogenetics, and designer receptors exclusively activated by designer drugs, have been developed in order to modulate and study the brain. Among these methods, optogenetics receives considerable attention for its capacity to selectively stimulate distinct cell-types with high spatial and temporal resolution. However, optogenetics heavily relies on methods such as transgenic approaches, viral vector transfection, or nanoparticle injection for deep brain application, which pose practical challenges for translation in human clinical utility. In contrast, non-invasive methods such as transcranial magnetic stimulation and transcranial current stimulation are readily translated to clinical utility, but are challenged to achieve highly spatial focus and deep penetration.

Unlike other noninvasive neuromodulation technologies such as transcranial magnetic stimulation and transcranial current stimulation, low-intensity tFUS can be applied in many neuromodulation applications due to its high spatial focality and its non-invasive nature. During tFUS neuromodulation, pulsed mechanical energy is transmitted through the skull with high spatial selectivity, which can be steered and utilized to elicit activation or inhibition through parameter tuning. Prior studies have investigated the neural effects of ultrasound parameters, such as ultrasound fundamental frequencies (UFF), intensities (UI), durations (UD), duty cycles (UDC), pulse repetition frequencies (UPRF), and other parameters. tFUS has been observed to induce behavioral changes, e.g. motor responses, electrophysiological responses, e.g. electromyography (EMG), electroencephalography (EEG), local field potentials (LFPs), and multi-unit activities (MUAs), with high in-vivo temporal/spatial measurement fidelity, or neurovascular activities, e.g. blood-oxygenation-level-dependent (BOLD) signal. To further achieve selectivity in stimulating brain circuits or even among cellular populations, focused ultrasound has been employed in combination with specific neuromodulatory drug-laden nanoparticles, cell-specific expression of ultrasound sensitizing ion channels, or acoustically distinct reporter genes in microorganisms. So far, prior studies have not explored the intrinsic effects of the wide range of ultrasound parameters on specific neuron subpopulations, such as regular-spiking and fast-spiking units.

The ability to selectively stimulate neural subpopulations non-invasively can provide a powerful scientific or clinical tool. For example, tFUS may be used to modulate atrophied brain regions in patients with Alzheimer disease to prevent disease progression or improve cognitive function. Studies have shown the Papez circuit in the anterior nucleus of the thalamus projects to multiple areas of the brain involving memory such as the dentate gyrus, anterior cingulate cortex, and frontal and temporal regions. Deep brain stimulation of these regions has been explored to help improve memory. Therefore, the innovation of embodiments of the present invention is methods and a system for application of transcranial focused ultrasound to target specific neuron populations and to elicit long-term changes in synaptic connectivity in the deep brain, allowing the delivery of long lasting therapy for clinical utility.

### BRIEF SUMMARY

According to one embodiment, specific neuron types are targeted non-invasively for neuromodulation by altering the tFUS pulse repetition frequency, where a dynamic acoustic radiation force is induced by the tFUS at the ultrasound pulse repetition frequency. In an alternative embodiment, the parameters of tFUS are chosen to encode temporally dependent stimulation paradigms into neural circuits and non-invasively elicit long-term changes in synaptic connectivity. In certain embodiments, a collimator is used to focus and direct ultrasound energy to specific areas of the brain.

### BRIEF SUMMARY OF THE SEVERAL VIEWS OF THE DRAWINGS

FIGS. 1A-1D show ultrasound stimulation at an angled or normal incidence, with detailed views of the collimators also shown.

FIG. 2 shows a diagram depicting an ultrasound temporal sequence.

FIG. 3 depicts ultrasound pressure distribution under the cranium using angled incidence.

FIG. 4 is a graph showing multi-unit activity with concurrent tFUS or sham conditions.

FIG. 5 is a chart showing various ultrasound parameters.

FIGS. 6A-6F show the response of different neural populations at different UPRF levels.

FIG. 7 depicts a tFUS stimulation waveform for long-term effect induction, according to one embodiment.

FIG. 8 depicts ultrasound pressure distribution under the cranium using normal incidence.

FIGS. 9A-9D are graphs showing responses to tFUS stimulation.

FIG. 10 depicts a tip-changeable ultrasound collimator.

FIG. 11 is a graph showing the normalized field excitatory postsynaptic potential slope at different ultrasound pulse repetition frequencies.

FIG. 12 shows a flexible EEG sensor.

FIGS. 13A-13D depict an ultrasound array.

FIG. 14 depicts multiple transducers to transmit ultrasound to the brain.

### DETAILED DESCRIPTION

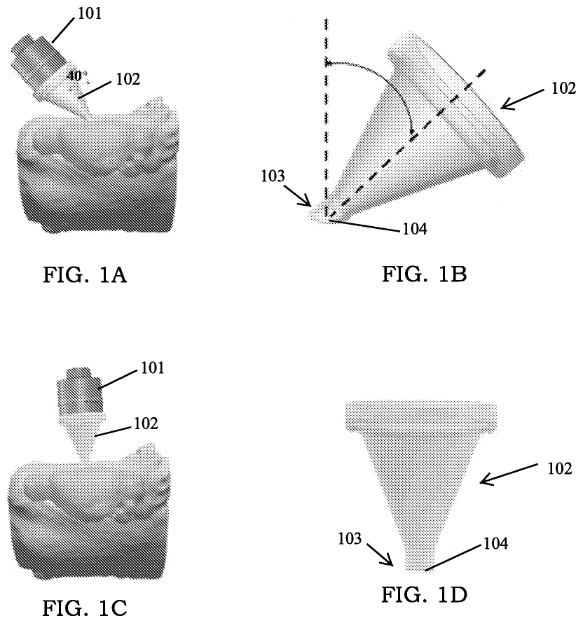

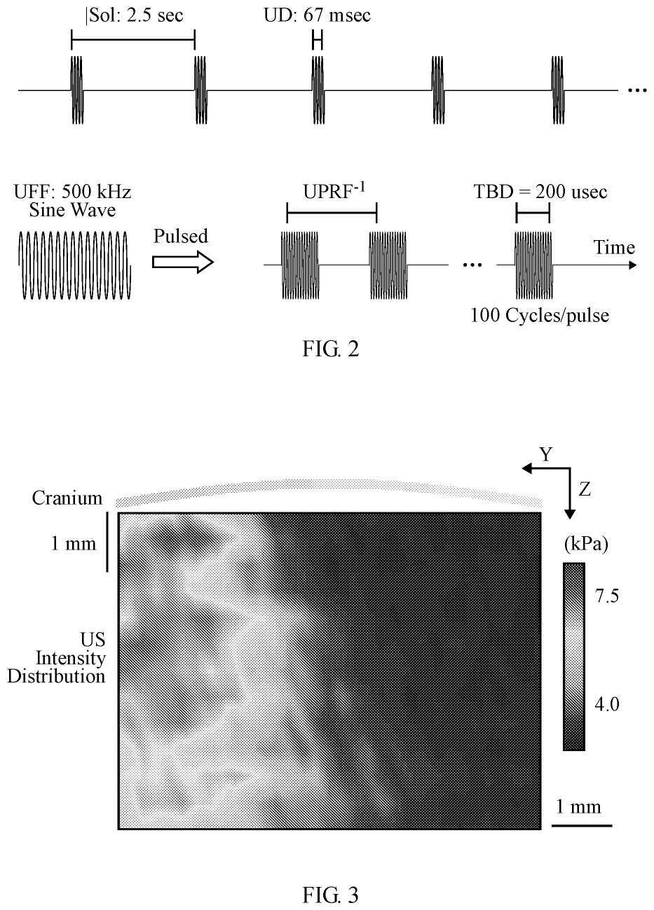

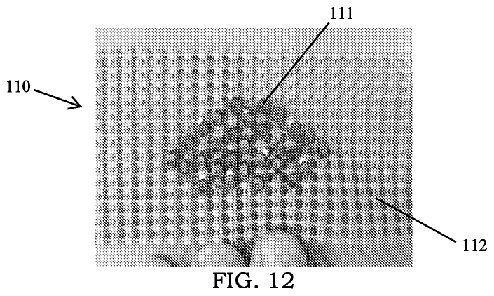

In one embodiment, a method of stimulating a response in specific neural populations comprises generating pulsed tFUS using a single-element transducer 101, guided to a scalp location over the cortex through a mounted 3-D printed collimator 102 filled with aqueous ultrasound gel at an incidence angle of 40°, as shown in FIG. 1A. A more detailed view of the collimator 102, having an angled tip 103, is shown in FIG. 1B. The precise location of tFUS stimulation is functionally monitored through a customized flexible EEG cap 110 (FIG. 12) that provides real-time feedback of neural activation in response to ultrasound stimulation. In alternative embodiments, the incidence angle varies from to normal to above 40° (±3 degrees) depending on the intended application. The stimulation dynamics of the tFUS waveforms can consist of tone-burst sinusoidal waves with constant UFF (500 kHz), and TBD (200 μsec), and varied UPRF (e.g. between 30-4,500 Hz). FIG. 2 is a graphical depiction of the ultrasound waveform. As shown in FIG. 2, the duration of inter-sonication interval (ISoI) is 2.5 seconds per trial. Referring to FIG. 3, an ultrasound spatial map from the coronal view (y-z plane) is shown, in which mechanical energy is distributed along a coronal beam up to a depth of 4 mm, but spatial peak energy is located within 1 mm from behind the skull. As FIG. 3 illustrates, the 40° incidence angle allows shallow targeting at the cortex, dissipating the majority of ultrasound energy through the skull. In this embodiment, angled tFUS stimulation is used as its activation pattern is shallower than normally incident tFUS.

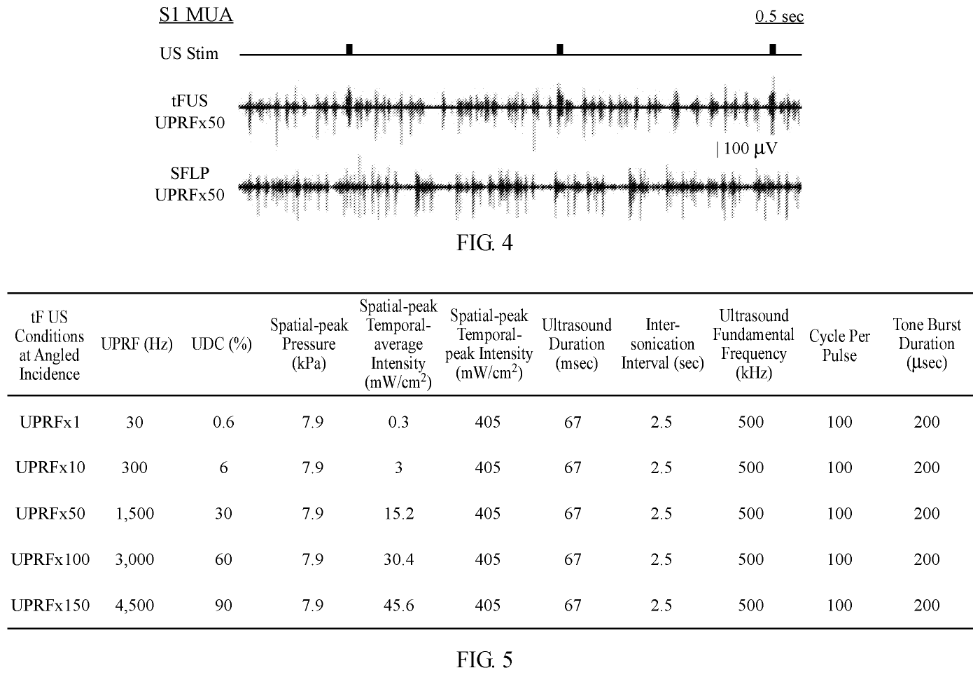

FIG. 4 is an example of acquired multi-unit activity (MUA) from the primary somatosensory cortex (Si) using tFUS with UPRF=1500 Hz. The timing between the ultrasound-induced action potentials and the administered stimulations are exemplified by 4 trials. The sham condition, in which tFUS with the parameters described above is not administered, SFLP shows a silence of such time-locked MUA. As shown in FIG. 4, during tFUS stimulation, increased time-locked neuronal firing is observed in recorded MUA as compared to SFLP sham conditions when the acoustic aperture of the collimator 102 is directed 180 degrees away from the target.

Cell-Type Selective Effects of tFUS

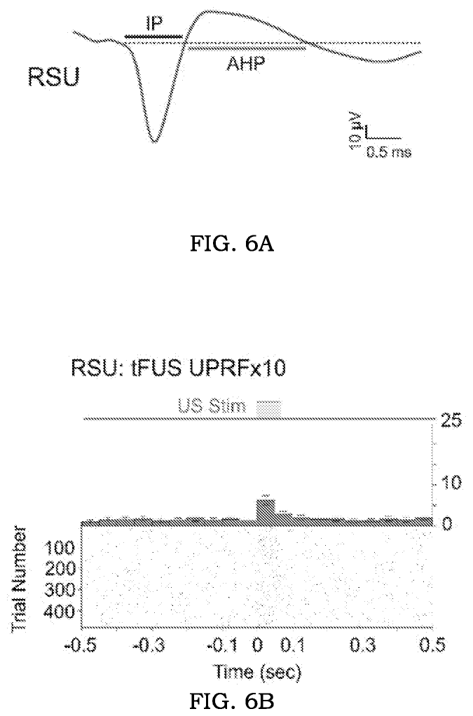

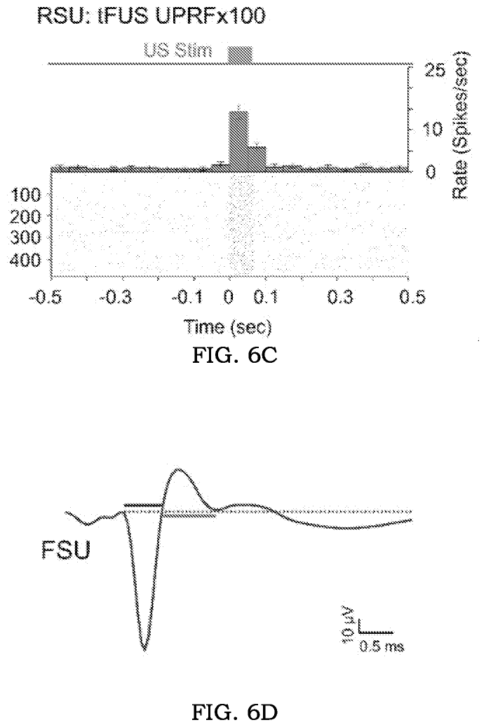

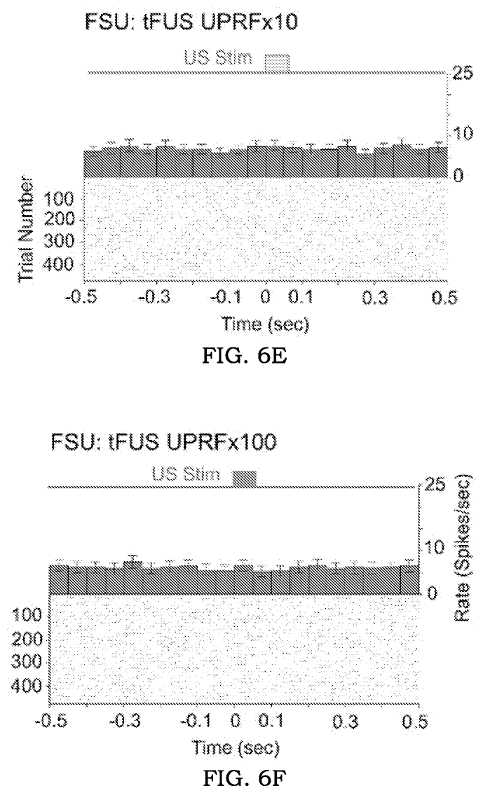

The fundamental unit for constructing the above ultrasound wave is the tone burst period which includes 100 cycles sinusoidal wave per pulse. The UPRF determines the durations between two consecutive ultrasound pulses. The sonication duration, tone burst duration (i.e. cycle per pulse number), the fundamental frequency, and ultrasound pressure magnitude, etc. can be used as shown in FIG. 5. The regular spiking units (RSU) are presumably considered as excitatory neurons with longer temporal signatures in the waveforms of action potentials than those of the fast spiking units (FSU, presumably as inhibitory neurons) as shown in FIGS. 6A and 6D. In the low UPRF, e.g. 300 Hz, the RSU has lower spiking rate compared to the FSU as shown in FIGS. 6B and 6E, although the RSU has slightly increased its own spiking rates comparing to that of the pre-stimulus period in FIG. 6B. While the high UPRF at 3000 Hz leads to a significant increase of the spiking rates for the RSU, but not for the FSU. The balance leaning towards inhibitory effects is transformed to excitatory effects due to the increase of UPRF. Thus, in this embodiment, the intrinsic cell-type selectivity in response to the deposited ultrasound energy to the brain occurs without introducing any external materials to the brain tissue.

All recorded action potentials from a 32-channel electrode array can be sorted based on the spike waveforms and inter-spike intervals (ISpI). The extracted features are the durations of initial phase (IP) of the action potential, i.e. from onset to the re-crossing of baseline, and afterhyperpolarization period (AHP), i.e. from the end of the IP to its re-crossing of baseline, shown in FIGS. 6A and 6D. The differences in these features have been associated with differences of ion channel types and distributions in the neuronal cell membrane. Thus, RSU and FSU will have distinct responses to various tFUS stimulation sequences due to their intrinsic cellular differences.

The neural effects of the administered pulsed tFUS can be confirmed through intracranial MUA recordings. Using peri-stimulus time histograms (PSTH), a significant increase of spike rate (6.23±1.10 spikes/sec) in a possible regular-spiking somatosensory cortical neuron (mean spike waveform IP: 0.85 ms, AHP: 1.8 ms) when stimulated with a tFUS condition (UPRF=300 Hz, Iₛₚₜₐ=3.0 mW/cm²) is observed, with a further increased spiking rate (14.35±1.65 spikes/sec) in response to the increase sonication (UPRF=3000 Hz, Iₛₚₜₐ=30.4 mW/cm²). For a more intuitive comparison, increased spike rate as a function of time along 478 consecutive trials are demonstrated with the raster plot (FIG. 6B-6C), in which the density of spiking events increases during the ultrasound stimulation. For another identified RSU, the comparisons are shown between the tFUS and sham conditions, i.e. flipping acoustic aperture 104 away from the brain or directing ultrasound energy to a control location over the scalp.

In contrast, a fast-spiking cortical neuron (FIG. 6D) with shorter durations of IP (mean: 0.7 ms) and AHP (mean: 0.65 ms) shows a more homogeneous PSTH distribution in response to the levels of tFUS treatment (e.g. UPRFx10 and UPRFx100 in FIG. 6E-6F) tested using the ultrasound setup shown in FIG. 1A. Return plots would illustrate a fast spiking behavior with a shorter refractory period than the RSU spiking unit identified in FIG. 6D. As seen in the example (FIG. 6E-6F), FSU firing rates are not disturbed by tFUS, i.e. that the firing rate (7.6±1.2 spikes/sec) is not significantly altered by the US stimulation (UPRF=300 Hz, Iₛₚₜₐ=3.0 mW/cm²) comparing to pre-stimulus rates (e.g. 7.5±1.2 spikes/sec at the bin of [−0.05, 0] s). For FSUs, no significant changes in spike rates are found (6.5±1.3 spikes/sec) even when ultrasound is administered at a UPRF 10 times higher (UPRF=3000 Hz, Iₛₚₜₐ=30.4 mW/cm², FIG. 6F). Furthermore, the return plots would indicate that although this fast-spiking neuron does not significantly change its rate of firing action potentials in response to tFUS, there is a possible trend of changing the spiking pattern of its bursting mode.

In a population level, the RSUs significantly increase their firing rates in response to UPRFs at 3000 and 4500 Hz when both comparing to that induced by a low UPRF at 30 Hz (UPRFx1 vs. UPRFx100: p=0.003; UPRFx1 vs. UPRFx150: p=0.0004). Whereas in the FSU group, no significant difference between tFUS conditions could be found. This implies that the spike rates of the FSUs are not significantly altered by the levels of UPRF.

The contrast between the responses observed in these two different neuron types suggests a cell-type selective mechanism by tFUS. The RSU group did not show significant differences among the five levels of sham ultrasound conditions.

Since the length of the refractory period determines the minimum time between neuronal firings, it follows that FSU spikes faster than the RSU did (the pre-stimulus firing rate as illustrated in FIGS. 6E-6F vs. FIG. 6B-6C). When administered with a low UPRF (i.e. 30 Hz), the RSUs do not respond significantly to tFUS stimulation, meanwhile FSUs also maintain a stable spiking state during the sonication. The observed responses suggest cortical neurons with different action potential shapes, hence different distribution of ion channels, in terms of ion channel types or relative quantity, have distinct response patterns to tFUS UPRF or duty cycle tabulated in FIG. 5. While FSUs are maintaining the activities across all UPRF frequencies, RSUs only exhibit increased firing rate during high UPRFs.

Long-Term Effects of tFUS

Beyond investigations on the short-term intrinsic effects of UPRF on neuron subtypes, the following method uses tFUS parameters for encoding frequency specific information into the brain for long-term effects. In this method, a specific tFUS temporal sequence and ultrasound pressure is delivered to the deep brain, e.g. the synaptic connections in hippocampus, to induce more than 30-minute sustained neural effects after the cessation of tFUS with minimal temperature rise at skull-brain interface and at the brain target.

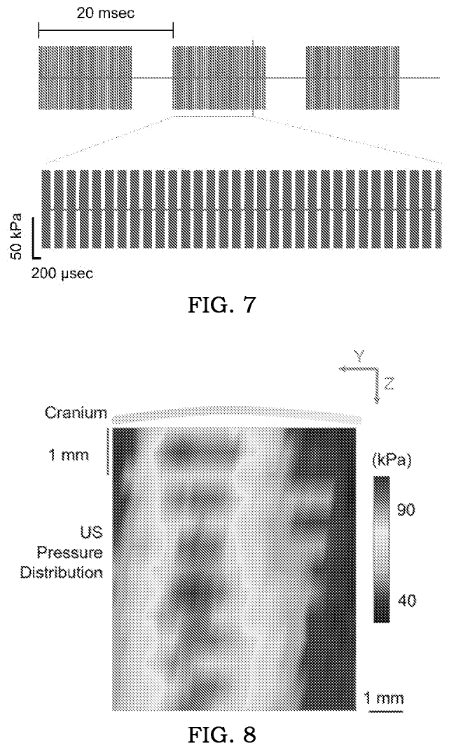

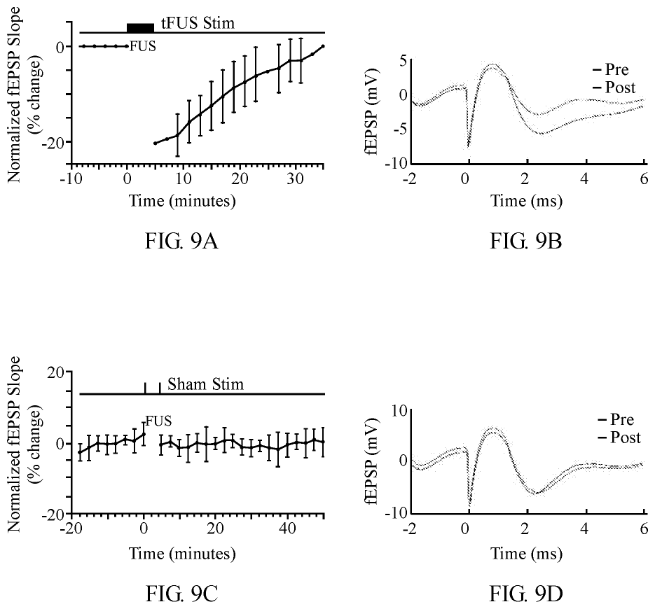

According to the method of this embodiment, the ultrasound spatial-peak pressure and UPRF are increased to 99 kPa and 3-10 kHz, and the inter-sonication interval is largely decreased to 20 msec, i.e. the inter-sonication frequency is increased to 50 Hz. FIG. 7 depicts the ultrasound waveform used in this embodiment. During the 20 msec period, 60% duty cycle is used. 5 minutes of repetitive sonication is used to evoke the change in the recorded field excitatory postsynaptic potential (fEPSP) effectively as shown in FIGS. 9A-9B using a comparison with sham ultrasound stimulation in FIG. 9C-9D. The aforementioned ultrasound parameter set provides an effective non-invasive approach for inducing long-term effects in changing the in-vivo hippocampal synaptic connections.

For a deeper brain target, another collimator 102 is used to allow normal incidence of tFUS at the scalp as shown in FIGS. 1C-1D. With this collimator 102, there is a much higher and deeper spatial-peak ultrasound pressure field (FIG. 8) as compared to using the angled incidence (FIG. 3). The ultrasound energy is able to penetrate deep and aim at the subcortical regions with the normal ultrasound incidence.

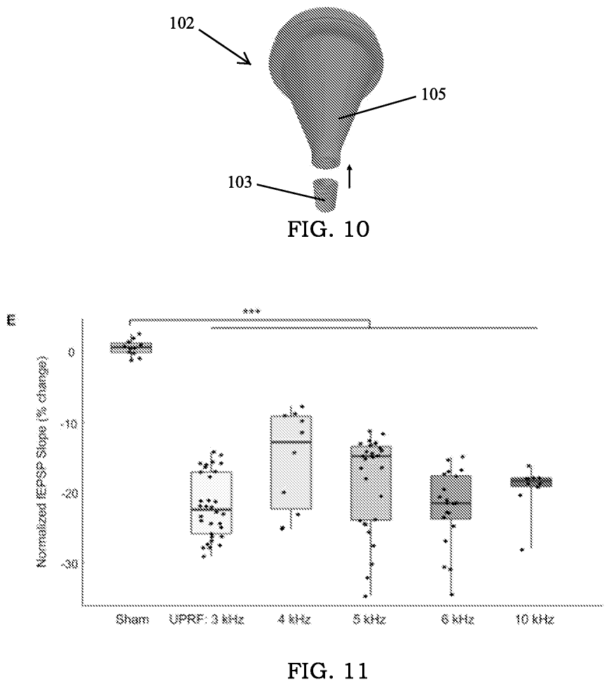

The ultrasound collimator 102, as shown in FIG. 10, can couple and guide ultrasound energy to a specific target. The 3D printed collimator 102 can be used for controlling the depth of ultrasound penetration in the living brain, thus physically limiting the ultrasound field within the superficial cortical brain, e.g. somatosensory cortex, without affecting the deep brain structure by using the angled incidence. The angled incidence is enabled by the angled collimator tip 103, and such tip 103 with different angles can be printed separately and be attached to one same collimator body 105 using mechanical threading in order to accommodate different application needs as shown in FIG. 10. This allows a user to selectively stimulate the cortical brain using focused ultrasound without affecting the deep brain area. For targeting deep brain structures, e.g. hippocampus, a smaller incidence angle, e.g. 30° or the normal incidence would allow the ultrasound to penetrate deeper. In one example embodiment, VeroClear™ is the base material of the 3D printed collimators, and its natural transparency will allow the eye examination of air bubbles existing in the coupling gel filled in the collimator 102 and the consequent removal of the bubbles using syringes thereafter.

To test whether tFUS can induce frequency encoded potentiation in the synapse, the induction of long-term potentiation (LTP) using pulsed tFUS in naïve rats was attempted using the method of the present invention. In the application of this example embodiment, pulsed tFUS stimulation was applied with various UPRFs at 50-100 Hz sonication frequency (FIG. 7) in order to emulate the effects of high frequency electrical stimulation of LTP in the dentate gyrus. Field response is assessed from the maximum descending slope of the fEPSP. Given that when the UPRF reaches 3 kHz, the cortical RSUs exhibit significant increased activities, the UPRF was set starting from 3 kHz for studying the transcranial neural effects at the deep brain. In the rats, instead of LTP, long-term depression (LTD) was observed in the fEPSP when tFUS stimulation was applied with UPRF of 3-10 kHz (FIG. 11). LTD was observed to persist 30 minutes after stimulation cessation. The fEPSP slope significantly decreases after tFUS stimulation and returns toward baseline over time. After all tFUS experiments, LTD is elicited using low frequency (1 Hz) electrical tetanus stimulation to validate the correct localization of neural pathway. Sham experiments with tFUS delivered at 180 degrees away from the skull demonstrate that LTD does not occur without the presence of tFUS. Averaged slope changes in fEPSP immediately pre or post tFUS stimulation, averaged across 5 minutes. The slope of the descending segment of fEPSP after the electrical pulse at 0 msec is used to calculate fEPSP slope.

It can be expected to observe LTP after tFUS stimulation since tFUS was applied at the same frequency as the high frequency tetanic stimulation used in certain prior art. However, the observed results did not show LTP, suggesting that the temporal encoding using tFUS does not share the same efficiency and/or mechanism as electrical tetanus stimulation. As such, the demonstrated long-term effect is a promising new feature of tFUS stimulation to be employed as a potential non-invasive therapeutic neuromodulation technique. The results suggest that tFUS can be used to encode time dependent stimulation paradigms into neural networks and non-invasively elicit long-term changes in the strength of synaptic connections.

In order to determine whether tFUS UPRF has an effect on strength of LTD induction, a range of UPRFs from 3 to 10 kHz were examined. As shown in FIG. 11, all of the UPRFs applied are significantly different when compared to sham simulation. Although there are no observed statistically significant differences between the sample groups, overall LTD can be observed across all sample groups. This suggests tFUS UPRF is not correlated with LTD, however the strength of LTD induction may be affected by tFUS UPRFs.

In the methods described above, single element focused transducers 101 were used for tFUS stimulation. In one embodiment, the transducer diameter is 28.5 mm with an ultrasound fundamental frequency (UFF) of 0.5 MHz, a −6 dB bandwidth at 300-690 kHz, and a nominal focal distance of 38 mm. For example, transducer model V391-SU-F1.5IN-PTF manufactured by Olympus Scientific Solutions Americas, Inc., USA can be used. Collimators 102 were 3D printed with VeroClear™ material to match the focal length of the transducer 101 and the animal model, the outlet or aperture 104 of the angled collimator 102 for the rat model has an elliptical area of 25.6 mm² (major axis length: 6.8 mm, minor axis length: 5 mm), and the one for the ultrasound normal incidence has a circular area of 19.64 mm². The size of collimators' outlet 104 was set to be no less than or at least commensurate with one ultrasound wavelength (i.e. 3 mm in soft tissue when using UPRF=500 kHz). One single-channel waveform generator can be used in connection with another double-channel generator to control the timing of each sonication, synchronize the ultrasound transmission with neural recording, and form the initial ultrasound waveform to be amplified, thus driving the transducer. A 50-watt wide-band radio-frequency (RF) power amplifier can be employed to amplify the low-voltage ultrasound waveform signal. The employed ultrasound intensity levels and duty cycles are described in FIG. 5. As noted in the table at FIG. 5, all ultrasound conditions used the same UFF of 0.5 MHz, ultrasound duration (UD, also known as sonication duration) of 67 msec, inter-sonication interval (ISoI) of 2.5 sec, tone-burst duration (TBD) of 200 μsec.

A single-element focused transducer 101 delivers tFUS stimulation at the dentate gyrus through the rat skull. The transducer 101 interfaces with the skull via a collimator 102 filled with ultrasound gel, with a tip diameter of 5 mm. 10-20 min of baseline fEPSP recorded before tFUS stimulation. Pulsed tFUS stimulation was delivered for 5 minutes at various UPRFs.

Referring again to FIG. 12, a flexible EEG cap is used to monitor the global brain response to the deposited ultrasound. In one embodiment, the electrodes 111 are adapted from commercially available pre-wired Ag/AgCl electrodes (electrode's impedance ranges from tens to hundreds of Ohms), then each electrode 111 is soldered and rewired to a thin and flexible wire. After soldered junctions are strengthened through adhesives, each of the rewired electrodes is glued to a gridded fabric substrate 112 as shown in FIG. 12. The grid size, spacing, and the coverage area of the EEG can be customized to accommodate different head sizes and geometrical features. Such a highly customization capability further allows dedicated openings for acoustic incidence window, i.e. insertion area for the ultrasound collimator's tip 103 and intracranial devices (if used), e.g. intracranial placement for recording electrodes, electrical and/or optical stimulations to the brain. The reserved space over the EEG cap can be occupied by the insertion pathway of intracranial recording electrodes, thus multi-scale electrophysiological recordings can be achieved in order to simultaneously capture both global and local brain activations in high temporal resolutions. Non-functional replicas of this cap can be easily made for micro CT imaging to colocalize electrode positions with anatomical landmarks, especially useful for EEG scalp action reconstructions.

In another embodiment, the said-EEG recordings over the scalp are used to localize and image brain electrical sources that are induced by ultrasound stimulation. This can be done by minimizing the difference between a source-model predicted scalp EEG and the recorded EEG over the scalp. The source distributions within such a brain are used to inform targets of focused ultrasound stimulation.

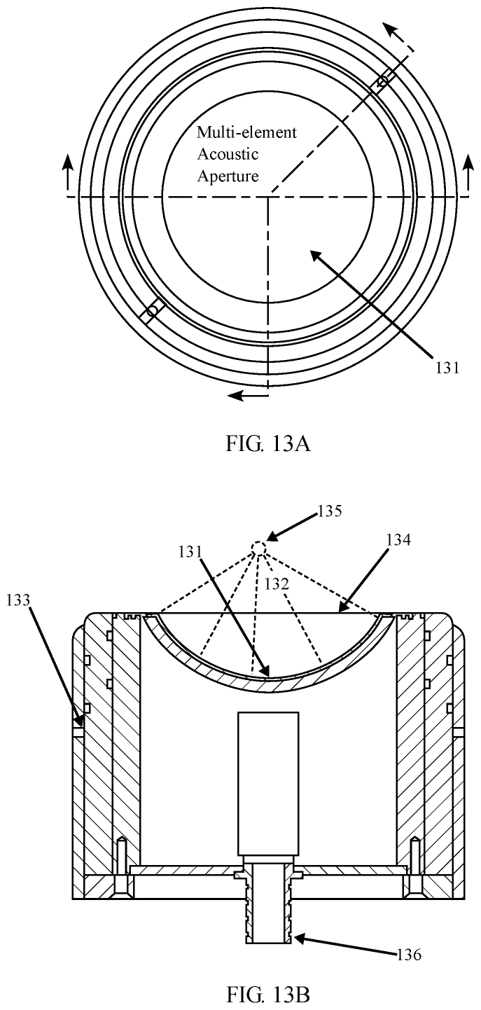

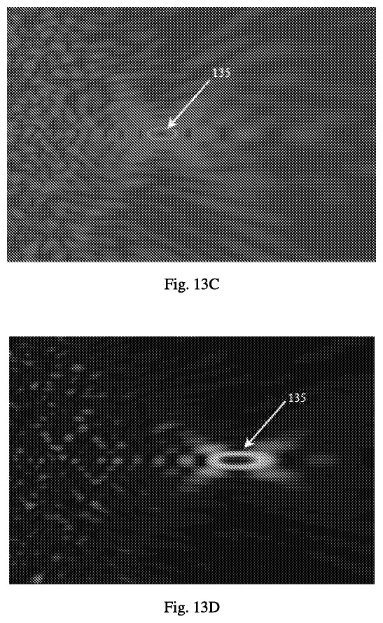

In another embodiment, the cell-type selectiveness and the long-term effects of tFUS can be delivered by multi-element (i.e. more than one element) tFUS stimulation at the cortical brain and deep brain areas, respectively. The multi-element transducer array shown in FIGS. 13A and 13B demonstrates its structure to achieve the ultrasound focus 135 through the acoustic exit plane 132. Coupling material is filled within the concave space between the element surface 131 and the interface 134 before the neuromodulation object. The case 133 of the multi-element ultrasound transducer can provide physical channels to external materials for coupling, recordings and intervention. The UPRF is controlled and delivered through the interfacing connection 136. As shown in FIGS. 13C and 13D, the ultrasound focus 135 can be electrically steered in space, e.g. various depths or lateral scanning. For example, in our embodiments of the disclosure, the shallow depth in FIG. 13C can be employed to achieve cell-type selective effects, while the deeper focus in FIG. 13D is able to achieve the long-term effects in specific neural system. The UPRF ranging from 30 Hz to 10 kHz is the key factor that is able to realize those neural effects with the assistance of multi-element ultrasound transducer for better focusing and steering. The above tFUS scheme can work independently without any collimator or coupling devices, but it is not preventing the potential use of collimator structures in FIGS. 1B and 1D for different application scenarios.

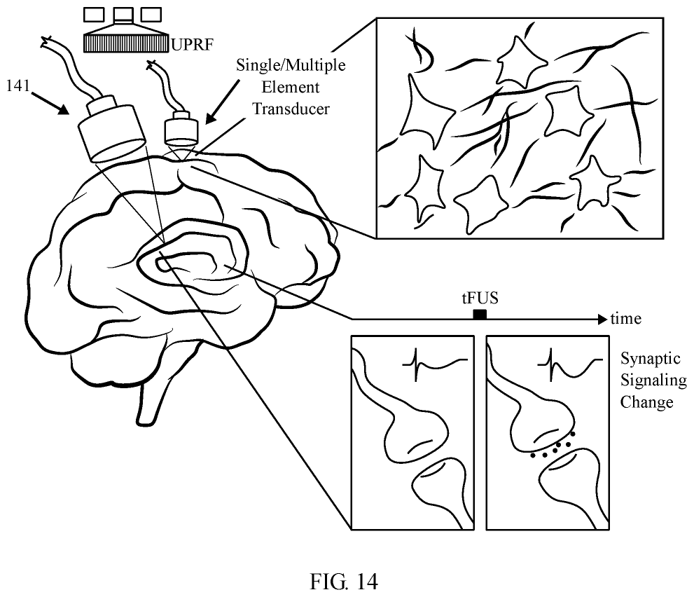

Overall, as shown in FIG. 14, the single/multiple element transducer 141 once fed with appropriate UPRF with certain focal depth control, the tFUS neuromodulation is able to achieve the cell-type selectiveness and/or long-term neural effects without introducing any external agents.

While the disclosure has been described in detail and with reference to specific embodiments thereof, it will be apparent to one skilled in the art that various changes and modification can be made therein without departing from the spirit and scope of the embodiments. Thus, it is intended that the present disclosure cover the modifications and variations of this disclosure provided they come within the scope of the appended claims and their equivalents.

What is claimed is:

1. A method of stimulating a response in neural populations within a brain using transcranial focused ultrasound comprising:
transmitting pulsed transcranial focused ultrasound through the cranium,
wherein the transcranial focused ultrasound comprises tone-burst waves with an ultrasound pulse repetition frequency of between 30 Hz and 10,000 Hz.

2. The method of claim 1, wherein the waves have a constant ultrasound fundamental frequency of between 200 kHz and 2,000 kHz and a tone burst duration of 200 microseconds.

3. The method of claim 1, wherein the pulsed transcranial focused ultrasound is transmitted using a transducer having a collimator positioned at a distal end.

4. The method of claim 1, wherein the tone-burst waves are sinusoidal.

5. The method of claim 1, further comprising:
monitoring the location of the pulsed transcranial focused ultrasound within the brain using scalp electroencephalography (EEG) recordings.

6. The method of claim 5, wherein the EEG recordings are made using a flexible EEG cap comprising:
a plurality of electrodes attached to a fabric substrate in a grid pattern.

7. The method of claim 1, wherein the collimator transmits the pulsed transcranial focused ultrasound through the cranium at an angle of incidence of about 40 degrees.

8. The method of claim 1, wherein the collimator transmits the pulsed transcranial focused ultrasound through the cranium at an angle of incidence of about 0 degrees.

9. The method of claim 1, wherein the collimator has an aperture with a diameter no less than a wavelength of the pulsed transcranial focused ultrasound.

10. The method of claim 1, wherein the tone-burst waves have 100 sinusoidal wave cycles per pulse.

11. The method of claim 1, wherein the pulsed transcranial focused ultrasound comprises tone-burst sinusoidal waves with an ultrasound pulse repetition frequency of about 1500 Hz.

12. The method of claim 1, wherein the pulsed transcranial focused ultrasound comprises tone-burst sinusoidal waves with an ultrasound pulse repetition frequency of about 3000 Hz.

13. The method of claim 1, wherein the pulsed transcranial focused ultrasound comprises tone-burst sinusoidal waves with an ultrasound pulse repetition frequency of about 4500 Hz.

14. The method of claim 1, wherein the pulsed transcranial focused ultrasound is transmitted into the brain without introducing any external materials into the brain.

15. The method of claim 1:
wherein the pulsed transcranial focused ultrasound are transmitted through a plurality of transducers.

16. A method of stimulating a long-term response in neural populations within a brain using transcranial focused ultrasound comprising:
transmitting pulsed transcranial focused ultrasound through the cranium,
wherein the transcranial focused ultrasound comprises tone-burst waves with an ultrasound pulse repetition frequency of between 1 Hz and 10 kHz, an inter-sonication interval of 1-100 milliseconds, and an inter-sonication frequency of 10-1000 Hz,
wherein the transcranial focused ultrasound produces a spatial-peak pressure of sub-mega Pa.

17. The method of claim 16, wherein the transcranial focused ultrasound is transmitted repeatedly over a 5 minute period.

18. The method of claim 16, wherein the transcranial focused ultrasound comprises tone-burst sinusoidal waves.

19. The method of claim 16, wherein the transcranial focused ultrasound is transmitted using a transducer having a collimator positioned at a distal end.

20. The method of claim 16, wherein the transcranial focused ultrasound is transmitted using a plurality of transducers.

21. A system of stimulating a response in neural populations within a brain using transcranial focused ultrasound comprising:
at least one transducer for transmitting pulsed transcranial focused ultrasound through the cranium,
wherein the transcranial focused ultrasound comprises tone-burst waves with an ultrasound pulse repetition frequency of between 1 Hz and 10,000 Hz.
